(19) United States
(12) Patent Application Publication
(10) Pub. No.: US 2002/0004205 A1
(43) Pub. Date: Jan. 10, 2002

(54) Method of investigating functional molecular interactions and reagents for use therein

(76) Inventors: Thomas G. Consler, Durham, NC (US); John G. Gray, Durham, NC (US); Marie A. Iannone, Durham, NC (US); Julie B. Stimmel, Durham, NC (US)

Correspondence Address: NEEDLE & ROSENBERG P C, 127 PEACHTREE STREET N E, ATLANTA, GA, 30303-1811, US

(21) Appl. No.: 09/821,984

(22) Filed: Mar. 30, 2001

Related U.S. Application Data
(60) Provisional application No. 60/193,286, filed on Mar. 30, 2000.

(51) Int. Cl. C12Q 1/68; G01N 33/53; G01N 33/537; G01N 33/543

(52) U.S. Cl. 435/6; 435/7.92; 436/518

(57) ABSTRACT

The present invention provides a multiplexed assay for analyzing complex molecular interactions. Thus, the invention relates to a method of identifying, in a single assay, the relative binding of two or more first members of a binding pair to one or more second members of a binding pair. The invention also relates to a method of identifying, in a single assay, the relative modulation, by a modulating agent, of binding between two or more binding pairs or between two or more binding complexes. Further provided are methods of screening, in a single assay, for an agent or agents with a selected binding profile or for an agent that modulates binding between two or more first members of a binding pair and one or more second members of a binding pair or the members of two or more binding complexes. The invention also relates to a set of microspheres coupled with a set of cofactors, wherein the microspheres are labeled with a label specific for each cofactor of the set.

### CROSS REFERENCE TO RELATED APPLICATIONS

This application claims the benefit of, and priority to, U.S. provisional Application Serial No. 60/193,826, filed Mar. 31, 2000, which status is pending and the entirety of which is incorporated herein by this reference.

### BACKGROUND OF THE INVENTION

1. Field of the Invention

This invention relates generally to a rapid and sensitive method of investigating molecular functional interactions in a multiplexed, high-throughput format. The invention has broad applicability in the pharmaceutical industry as a means for identifying compounds useful in diagnosis or therapy.

2. Background Art

Molecules typically function by interaction with other molecules, including protein/protein interactions, nucleic acid/protein interactions, and nucleic acid/nucleic acid interactions. Furthermore, these functional interactions are frequently modulated by an additional molecule or molecules to form a macromolecular functional unit. Assays that have traditionally been used to evaluate these functional interactions include enzyme activity assays, ligand binding assays, endpoint assays, kinetic binding assays, competitive binding assays, hybridization reactions, BIACORE, homogeneous time-resolved fluorescence, scintillation proximity assays, and others.

Recently, flow cytometry has been used in simultaneous and multiplexed diagnostics and genetic analysis of clinical specimens (See WO 99/36564). Sets of microspheres marked with specific fluorescent dyes and having specific fluorescent profiles, available commercially from Luminex Corporation (Austin, Tex.), have been used for these purposes. Specific molecular entities were coupled to the microsphere surface so that each set had a different molecular entity coupled to it. Employing flow cytometric methodology, an integrated system of fluidics, optics, and electronics was used to evaluate binding to the coupled entities. Neither the traditional assays nor flow cytometry, however, have been used for detection of multiple interactions in a single-step assay as a means of studying functional hierarchical interactions in a group or as a means of screening for agents having a specific role in complex molecular interactions.

### SUMMARY OF THE INVENTION

In accordance with the purpose(s) of this invention, as embodied and broadly described herein, this invention, in one aspect, relates to a method of identifying, in a single assay, the relative binding of two or more first members of a binding pair to one or more second members of a binding pair, comprising (a) contacting the first members with the second member or members under conditions that allow the first members to bind with the second member or members to form binding pairs, wherein the first members are coupled directly or indirectly to a set of microspheres labeled with a set of first labels, wherein each first label is specific for one first member, wherein each second member is coupled directly or indirectly to a second label, and wherein each second label is specific for one second member; (b)detecting the presence of the first specific labels and the presence or absence of the second labels in a plurality of detection products, wherein each detection product comprises one microsphere; (c) comparing the relative amount of each second label associated with each first label in the detection products, wherein a greater or lesser amount of each second label associated with each first label in the detection product indicates first members, identified by the specific first label, having greater or lesser binding to the second member or members, indicated by the second specific label; and (d) creating a binding profile for each second member, the binding profile indicating the relative preference of binding between each first member and each second member of the binding pairs.

In another aspect, the invention relates to a method of identifying, in a single assay, the relative modulation, by a modulating agent, of binding between two or more binding pairs, comprising (a) contacting two or more first members and one or more second members of the binding pairs and the modulating agent, under conditions that allow formation of binding pairs; wherein the first members are coupled directly or indirectly to a set of microspheres labeled with a set of first labels; wherein each first label is specific for one first member; wherein the second member is coupled directly or indirectly to a second label; and wherein the second label is specific for one second member; (b) detecting the presence of the first specific labels and the second specific labels in a plurality of detection products, wherein each detection product comprises one microsphere; and (c) comparing, to a control, the relative amount of each second label associated with each first label in the detection products, wherein the control lacks the modulating agent, a greater or lesser amount of each second label associated with each first label in the detection product, indicating that the modulating agent potentiates or attenuates binding between the first member, indicated by the first specific label, and the second member, indicated by the second specific label.

In yet another aspect, the invention relates to a method of identifying, in a single assay, the relative modulation, by a modulating agent, of binding between the members of two or more binding complexes, comprising (a) contacting two or more first members, one or more second members, a third member of the binding complexes, and the modulating agent, under conditions that allow formation of binding complexes, wherein each binding complex comprises one first and one second member of the binding complex; wherein the first members are coupled directly or indirectly to a set of microspheres labeled with a set of first labels; wherein each first label is specific for one first member; wherein the second member is coupled directly or indirectly to a second label; and wherein the second label is specific for one second member; (b) detecting the presence of the first specific labels and the presence or absence of the second specific labels in a plurality of detection products, wherein each detection product comprises one microsphere; (c) comparing, to a control, the relative amount of each second label associated with each first label in the detection products, wherein the control lacks the modulating agent, a greater or lesser amount of each second label associated with each first label in the detection product, indicating that the modulating agent potentiates or attenuates binding between the first member, indicated by the first specific label, and the second member, indicated by the second specific label, in the presence of the third member.

In another aspect, the invention relates to a method of screening, in a single assay, for an agent or agents with a selected binding profile, comprising (a) contacting the agent or agents to be screened with two or more putative binding members under conditions that allow the agent or agents to bind with the binding members to form binding pairs, wherein the binding members are coupled directly or indirectly to a set of microspheres labeled with a set of first labels, wherein each first label is specific for one binding member, wherein each agent to be screened is coupled directly or indirectly to a second label, and wherein each second label is specific for one agent to be screened; (b) detecting the presence of the first specific label and the presence or absence of second specific labels in a plurality of detection products, wherein each detection product comprises one microsphere; (c) comparing the relative amount of each second label associated with each first label in the detection products, wherein a greater or lesser amount of each second label associated with each first label in the detection product indicates an agent, indicated by the second specific label, having greater or lesser binding to the binding members, indicated by the first specific label; (d) creating a binding profile for each agent to be screened, wherein the binding profile indicates the relative preference of binding between each agent to be screened and each binding member; and (e) comparing the binding profile for the agent or agents to be screened with the selected profile, the same binding profile indicating an agent with the selected binding profile.

In yet another aspect the invention relates to a method of screening, in single assay, for an agent that modulates binding between two or more first members of a binding pair and one or more second members of a binding pair, comprising (a) contacting two or more first members and one or more second members of the binding pairs and the modulating agent, under conditions that allow formation of binding pairs; wherein the first members are coupled directly or indirectly to a set of microspheres labeled with a set of first labels; wherein each first label is specific for one first member; wherein the second member is coupled directly or indirectly to a second label; and wherein the second label is specific for one second member; (b) detecting the presence of the first specific labels and the presence or absence of second specific labels in a plurality of detection products, wherein each detection product comprises one microsphere; (c) comparing, to a control, the relative amount of each second label associated with each first label in the detection products, wherein the control lacks the agent to be screened, a greater or lesser amount of each second label associated with each first label in the detection products, indicating an agent that modulates binding between the specific first member, identified by the specific first label, and the specific second member, identified by the specific second label.

In another aspect the invention relates to a method of screening, in a single assay, for an agent that modulates binding between the members of two or more binding complexes, comprising (a) contacting two or more first members, one or more second members, and a third member of the binding complexes with the agent to be screened, under conditions that allow formation of binding complexes, wherein each binding complex comprises one first and one second member; wherein the first members are coupled directly or indirectly to a set of microspheres labeled with a set of first labels; wherein each first label is specific for one first member; wherein the second member is coupled directly or indirectly to a second label; and wherein the second label is specific for one second member; (b) detecting the presence of the first specific labels and the presence or absence of second specific labels in a plurality of detection products, wherein each detection product comprises one microsphere; and (c) comparing, to a control, the relative amount of each second label associated with each first label in the detection products, wherein the control lacks the agent to be screened, a greater or lesser amount of each second label associated with each first label in the detection products, indicating an agent that modulates binding between the specific first member, identified by the specific first label, and the specific second member, identified by the specific second label, in the presence of the third member.

Yet another aspect of the invention relates to a set of microspheres coupled with a set of cofactors, wherein the microspheres are labeled with a label specific for each cofactor of the set.

The invention offers distinct advantages over the prior art in allowing multiplexed analysis of complex binding interactions. Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate embodiments of the invention and together with the description, serve to explain the principles of the invention.

FIG. 1 shows a complex binding profile of estrogen receptor (ER) β ligand binding domain (LBD) and peoxisome proliferator-activated receptor (PPAR) γ LBD for three LXXLL-motif coactivator peptides.

FIG. 2 shows a complex binding profile for ERβ LBD binding to 34 coactivator peptides in the presence or absence of estradiol (2 μM).

FIG. 3 shows a complex binding profile for ERβ LBD binding to 37 coactivator peptides in the presence or absence of tamoxifen (500 nM) or raloxifene (500 nM).

FIG. 4A shows a complex binding profile of coactivator peptides for LBDs derived from Receptor B (100 nM). FIG. 4B shows a complex binding profile, in the presence or absence of a compound designated Agent 1 (200 nM), of coactivator peptides for LBDs derived from Receptor C (100 nM). FIG. 4C shows a complex binding profile of coactivator peptides for PPAR γ LBD (100 nM) in the presence and absence of five different compounds (designated Agent 2, Agent 3, Agent 4, Agent 5, and Agent 6) at 1 μM.

FIG. 5 shows a complex binding profile of binding ERβ LBD with coactivator peptides (SRC-1(3(735-759), SRC-1(626-642), and SRC-1(2)(676-700) and a corepressor peptide(SMRT) (2329-2354) in the absence and presence of various concentrations of estradiol (50, 100, 150, 200 nM).

FIG. 6 shows the binding profile of ER α LBD to 34 different coactivator peptides in the absence or presence of estradiol, raloxifene or tamoxifen.

### DESCRIPTION OF THE PREFERRED EMBODIMENTS

The present invention may be understood more readily by reference to the following detailed description of preferred embodiments of the invention and the Examples included therein and to the Figures and their previous and following description.

As used in the specification and the appended claims, the singular forms “a,” “an” and “the” include plural referents unless the context clearly dictates otherwise. Thus, for example, reference to “a first member of a binding pair” includes mixtures of first members of binding pairs, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The invention provides a method of identifying, in a single assay, the relative binding of two or more first members of a binding pair to one or more second members of a binding pair, comprising the steps of (a) contacting the first members with the second member or members under conditions that allow the first members to bind with the second member or members to form binding pairs, wherein the first members are coupled directly or indirectly to a set of microspheres labeled with a set of first labels, wherein each first label is specific for one first member, wherein each second member is coupled directly or indirectly to a second label, and wherein each second label is specific for one second member; (b) detecting the presence of the first specific labels and the presence or absence of the second specific labels in a plurality of detection products, wherein each detection product comprises one microsphere; (c) comparing the relative amount of each second label associated with each first label in the detection products, wherein a greater or lesser amount of each second label associated with each first label in the detection product indicates first members, identified by the specific first label, having greater or lesser binding to the second member or members, indicated by the second specific label; and (d) creating a binding profile for each second member, the binding profile indicating the relative preference of binding between each first member and each second member of the binding pairs.

As used throughout, “in a single assay” means a single assay volume (e.g., a single tube or well). The single assay can be a multiplexed assay in which simultaneous, or near simultaneous, determinations of binding events can be measured from the same assay process. For example, numerous first members, second members, and additional members of numerous binding complexes can be added to the same assay process for subsequent identification and analysis. Preferably, this multiplexed format allows a washless format, which improves sensitivity, saves reagents, and promotes efficiency.

“Relative binding” means the amount of binding that occurs between one binding pair as compared to another binding pair. For example, one first member may have relatively higher binding to one specific second member than to a different second member. One first member may have higher binding to one second member as compared to the binding between a different first member and a different second member. Similarly, a specific first and a specific second member may have higher relative binding compared to a different first member and different second member. The amount of binding can include the absence of binding.

By “conditions that allow binding” of first members to second member or members, agents to be screened, or modulating agents to form binding pairs or complexes means conditions in which at least some first members can bind to some second members. Such conditions can also include conditions in which some bound first members/second members bind to third members, modulating agents, or agents to be screened. Such conditions do not require that all first and second members combine, only that the conditions allow for specific binding interactions to occur. These conditions include, for example, pH, time, temperature, and buffer composition, which allow binding between the members of interest.

By “binding pairs” is meant at least two binding members that have bound together. A modulating agent may bind to either member of the binding pair and may prevent or enhance binding between the first and second members of the binding pair. By “binding complex” is meant at least a binding pair and, optionally, a third member and/or a modulating agent. In some cases the modulating agent may allow binding between two members of a binding pair that do not form a binding pair in the absence of the modulating agent. Modulating agents optionally are identified or classified using peptide sequences that have been identified by phage display. Such peptide sequences can be used as receptor conformation-sensing sequences, which are then used to screen for modulating agents.

As used throughout, the “contacting” step of the present invention is preferably in vitro.

As used throughout, a “detection product” comprises one microsphere. Each microsphere can have a bound first member which can bind to a second member, a third member, an agent to be screened, or a modulating agent. The microspheres can be identified based on the first label.

The “first label” can include one or more labels mixed together that together constitute a specific “first label” for a subset of microspheres. For example, it is well known in the art that microspheres can be labeled with two or more flurochromes mixed together in varying concentrations, such that each specific label has a specific concentration of each fluorochrome. It is the specific concentrations of the various flurochromes together to provide a spectrum of labels that can be used to distinguish the various subsets of labeled microspheres.

One microsphere preferably has only one first member coupled to it, but a plurality of the same first members is preferably coupled to a single microsphere. If a second member or a labeled agent to be screened binds to at least one first member coupled to the microsphere, then a detection product can comprise the microsphere, at least one first member, and at least one second member or agent to be screened. Preferably, a plurality of second members or agents to be screened, which are the same or different than each other but which have specific second labels, bind to the numerous molecules of the first member. Detection products can also comprise additional members (i.e., third members, fourth members, fifth members, etc.) or additional modulating agents (including, for example, first modulators that reduce binding between the first and second members or second modulators that are agonists or antagonists (or modulators) of the first modulators).

By “a set of microspheres” is meant a group of microspheres consisting of subsets of microspheres labeled with distinguishable labels. By coupling each subset to a specific first member, or a plurality of the same first member, a specific label for each first member species can be detected. A known amount of coupled microspheres, and consequently a known amount of each first label, is added to each assay process. The first label for uncoupled microspheres is detectable and constant for each set. Thus, it is the relative amount of second label as compared to first label in a given detection product that is relevant as the amount of second label varies depending upon binding between the first and second members of the detection product.

“Coupled directly or indirectly” will be understood by one skilled in the art to include various methods for coupling. For example, the microspheres can be coated with strepavidin or maleimide or can be carboxylated, or any modification of strepavidin, maleimide, or carboxylation, and the first members to be coupled to these microspheres can be biotintylated, have one or more free amine groups, or have one or more free sulfhydryl groups, respectively. One skilled in the art would recognize that other coupling agents can be used. See, e.g., WO 99/19515 and WO 99/37814, which are incorporated herein by reference in their entirety for types of functional groups that can be used for coupling the first agent to the microspheres. Optionally, a linker can be used between the microsphere and the coupling agent.

Labels can also be coupled to the microspheres, agents to be screened, or second members using a variety of methods known in the art. As used throughout, “label” refers to a moiety (e.g., a hapten) that provides a means for labeling, as well as radiolabels, and fluorescent labels. Thus, the first and second labels can be radiolabels, dyes, fluorescent labels, or a combination thereof. Preferably, the microspheres contain the first labels. Alternatively, the label could be attached to the surface of the microsphere. The second label can be attached to the second member of a binding pair or complex or agent to be screened either directly or indirectly. For example, a fluorescent compound (or fluorochrome), such as fluorescein, can be incorporated into the member or agent to be screened. Alternatively, the second member or agent to be screened can be biotintylated and a subsequent detectable label like a fluorescently labeled strepavidin can be used to indirectly label the second member or agent to be screened. When the microspheres are coated with strepavidin and the second member or agent to be screened contains biotin, binding between the strepavidin on the microsphere and the biotin on the second member or agent to be screened can be avoided by saturating the system with free biotin.

One skilled in the art would recognize that numerous fluorochromes could be used as labels in the present method. See, e.g., WO 99/19515 and WO 99/37814, which are incorporated herein in their entirety for types of fluorescent dyes and fluorochromes that can be used as labels.

One skilled in the art, however, would recognize that a variety of types of microspheres can be used. See, e.g., WO 99/19515 and WO 99/37814, which are incorporated herein in their entirety for types of microspheres and methods of making and using same. For example, the microspheres can be polystyrene-divinylbenzene microspheres.

By “detecting the presence” of a label means detecting any amount of label. Thus, in some cases the amount of label may be an absence of the label.

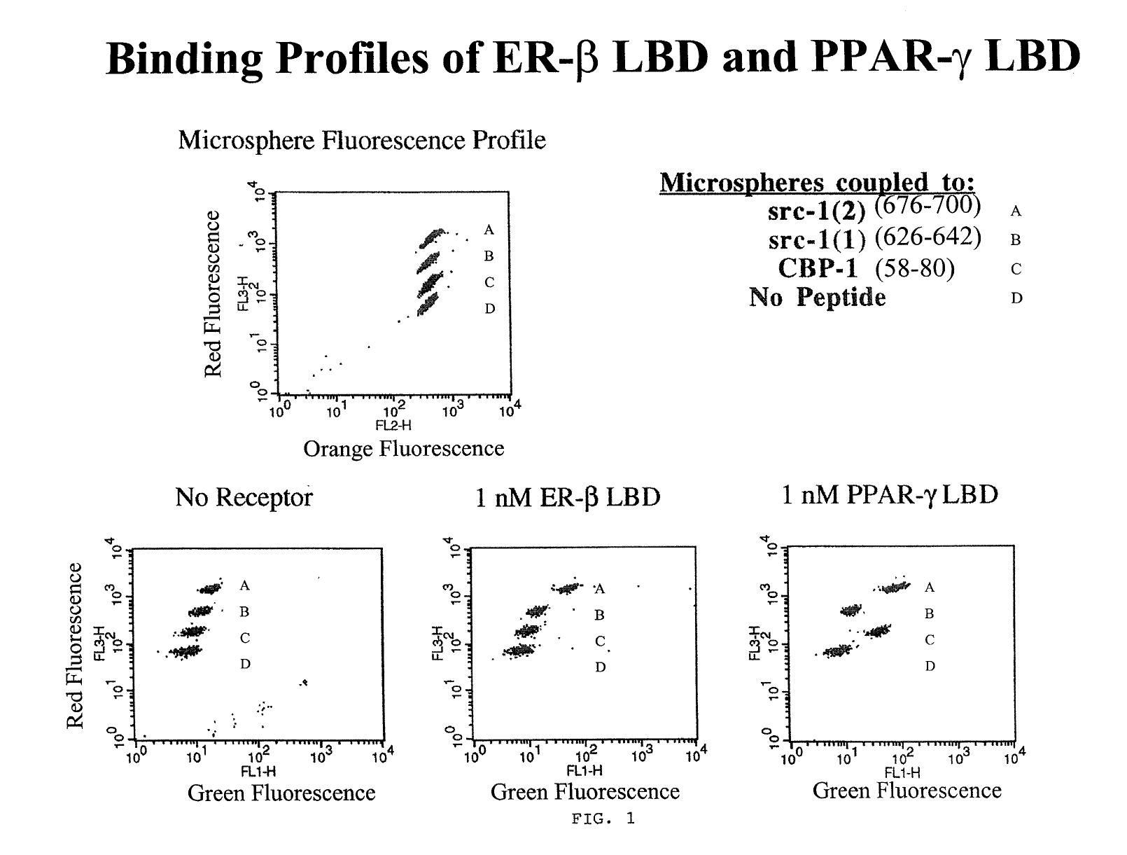

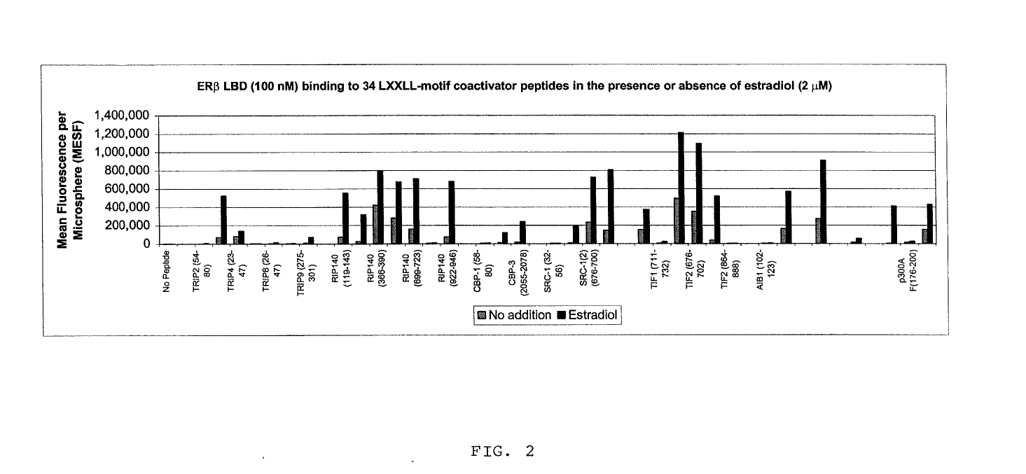

As used throughout, “a binding profile” is a systematic summary of the binding data such that the binding profile indicates the relative binding between each first member and each second member of various binding pairs. Thus, a specific binding profile indicates the hierarchical binding of each first member with each second member as compared to different first members and the same or different second members. The binding hierarchy can be a function of both K_{D} or Vₘₐₓ for each specific binding pair or binding complex. The data constituting a binding profile can be shown in several different ways. For example, the fluorescence profile can be shown, wherein a shift in the fluorescence profile of a subset of microspheres indicates the presence of a second label. See, for example, FIG. 1. Alternatively, the binding profile can be represented by plotting the mean fluorescence per microsphere as mean fluorescence index (MFI) or molecules of equivalent soluble fluorochrome (MESF) for each binding pair and/or each binding complex.

In the present invention, the first and second labels are excited by one or more light sources and detected using a detection device, wherein the detection device is capable of detecting and distinguishing each detection product and each label in the same detection product. Preferably, the light source or sources are lasers. Preferably, the labels are fluorescent dyes or molecules that can be detected and distinguished from all other first and second labels used in the assay. The first and second labels can be detected and distinguished by passing the microspheres across or through the light emitted by the light source or light sources. Alternatively the first and second labels are detected and distinguished by passing the emitted light across or through the microspheres. The microspheres can be on a two-dimensional surface, or traditional flow cytometric methods can be used, whereby the microspheres are aligned so that they travel in single file while being hydrodynamically restricted to the central part of a thin column of fluid. The rapidly moving line of microspheres are then passed before a focused beam of laser light, which excites the fluorochromes (i.e., first and second labels) associated with the microspheres. The laser excites the fluorochromes and the emitted fluorescent light is collected for each particle passing the beam, whereby different wavelengths are analyzed simultaneously or nearly simultaneously. The fluorescent light associated with each microsphere is reported in real time and/or stored and analyzed for characteristics that identify and quantify both the microsphere/first member and the second member/agent to be screened. Multiple fluorescence measurements can be made using the methods described in WO 98/59233, WO 99/37814, and WO 99/36564 which are incorporated herein in their entirety.

As used in the present invention, the first members are selected from a group consisting of cofactors, receptors, receptor ligands, proteins, peptides, protein domains, oligonucleotides, transcription factors, nucleic acids, small molecules, and small compounds, any of which can be synthetic, modified, or naturally occurring. The second members are selected from a group consisting of cofactors, receptors, receptor ligands, proteins, peptides, protein domains, oligonucleotides, transcription factors, nucleic acids, small molecules, and small compounds, any of which can be synthetic, modified, or naturally occurring. Thus, for example, the first members can be cofactors and the second member or members can be receptors. Optionally, the receptors can be coupled to the microspheres and the cofactors can be in solution.

By “small molecules” is meant natural or synthetic organic molecules less than about 1000 daltons and, more preferably, less than about 500 daltons. Small molecules, for example, include estradiol, cyclic nucleotides, retinoic acid, steroid hormones, amino acids, neurotransmitters (e.g., norepinephrine, epinephrine, acetylcholine), and numerous other compounds.

The receptors can be, for example, orphan receptors or nuclear receptors. As used herein, “orphan receptors” are molecules identified as having receptor or receptor-like domains or tertiary structures but lacking a known function. Thus, the present methods can be used to characterize the binding profile of an orphan receptor in an effort to characterize the function of the receptor. Nuclear receptors include, for example, all known receptors identified by the Nuclear Receptors Nomenclature Committee, 1999. See Vincent Laudet, Johan Auwerx, Jan-Ake Gustafsson, and Walter Wahli; A Unified Nomenclature System for the Nuclear Receptor Superfamily, Cell (1999) 97: 161-163, which is incorporated herein in its entirety by reference for the identification of known nuclear receptors. Nuclear receptors also include, for example, receptors that have yet to be identified but have at least one function or have at least one characteristic of known nuclear receptors.

“Cofactors” or “coregulators” as used throughout can refer to coactivators, corepressors, or a combination of coactivators and corepressors. Examples of nuclear receptor cofactors include, for example, those identified in Daniel Robyr, Alan P. Wolffe and Walter Wahli, Nuclear Hormone Receptor Coregulators in Action: Diversity for Shared Tasks, Mol.Endocrinol. (2000) 14: 329-347, which is incorporated herein in its entirety for examples of nuclear receptor cofactors.

As one embodiment, the invention can be used to confirm the results of a phage display that identifies amino acid sequences that bind to form a binding pair or binding complex. Thus the method comprises the steps of (a) expressing the amino acids to form the first members of a binding pair or binding complex; (b) coupling the first members to microspheres labeled with first specific labels, wherein each first label is specific for one first member; (c) contacting the first members with one or more second members, and optionally with a third member of a binding complex, under conditions that allow formation of binding pairs or binding complexes, wherein each second member is coupled directly or indirectly to a second label, and wherein the second label is specific for each second member; and (d) detecting the presence of the first specific labels and the second specific labels in a plurality of detection products, wherein each detection product comprises one microsphere. As an alternative embodiment, the invention can be used to confirm the results of a phage display that identifies amino acid sequence of two or more modulating agents of a binding pair or a binding complex

Also provided by the present invention is a method of identifying, in a single assay, the relative modulation, by a modulating agent, of binding between two or more binding pairs, comprising (a) contacting two or more first members and one or more second members of the binding pairs and the modulating agent, under conditions that allow formation of binding pairs, wherein the first members are coupled directly or indirectly to a set of microspheres labeled with a set of first labels; wherein each first label is specific for one first member; wherein the second member is coupled directly or indirectly to a second label; and wherein the second label is specific for one second member; (b) detecting the presence of the first specific labels and the presence or absence of the second specific labels in a plurality of detection product, wherein each detection product comprises one microsphere; (c) comparing, to a control, the relative amount of each second label associated with each first label in the detection products, wherein the control lacks the modulating agent, a greater or lesser amount of each second label associated with each first label in the detection product, indicating that the modulating agent potentiates or attenuates binding between the first member, indicated by the first specific label, and the second member, indicated by the second specific label.

By “relative modulation” is meant either attenuation or potentiation in the amount of binding that occurs between one binding pair in the presence or absence of a modulator as compared to the amount of binding that occurs in another binding pair in the presence or absence of the modulator. For example, one first member may have relatively higher binding to one specific second member in the presence of the modulator as compared to binding to the same second member in the absence of the modulator. This modulation may differ when the same first member binds to a different second member or when a different first member binds to the same or a different second member. Similarly, a specific first and a specific second member may have higher relative binding compared to a different first member and different second member in the presence of a modulator.

In one embodiment, the present method can further comprise creating a modulating profile for each modulating agent, wherein the modulating profile indicates the relative modulation of binding between each first member and each second member of the binding pairs. By “modulating profile” is meant a systematic summary of the binding data such that the modulating profile indicates the relative modulation by the modulating agent of binding between each first member and each second member of various binding pairs. Thus, a specific modulating profile indicates the hierarchical modulation of binding of each first member with each second member as compared to different first members and the same or different second members. The modulating profile may show that a particular modulating agent strongly attenuates binding between certain members of a binding pair but weakly potentiates binding between members of a different binding pair.

The modulating agent is selected from a group consisting of cofactors, receptors, receptor ligands, proteins, peptides, protein domains, oligonucleotides, transcription factors, nucleic acids, small molecules, and small compounds, any of which can be synthetic, modified, or naturally occurring. In one embodiment the first members are cofactors, the second member or members are receptors, and the modulating agent is a receptor ligand (e.g., estradiol).

Complex functional interactions of binding complexes (i.e., having more members than just a binding pair) can be assessed using the present invention. Thus, the present invention also provides a method of identifying, in a single assay, the relative modulation, by a modulating agent, of binding between the members of two or more binding complexes, comprising (a) contacting two or more first members, one or more second members, a third member of the binding complexes, and the modulating agent, under conditions that allow formation of binding complexes, wherein the binding complex comprises one first and one second member, and optionally, a third member and/or a modulating agent; wherein the first members are coupled directly or indirectly to a set of microspheres labeled with a set of first labels; wherein each first label is specific for one first member; wherein the second member is coupled directly or indirectly to a second label; and wherein the second label is specific for one second member; (b) detecting the presence of the first specific label and the presence or absence of a second specific label in a plurality of detection products, wherein each detection product comprises one microsphere; (c) comparing, to a control, the relative amount of each second label associated with each first label in the detection products, wherein the control lacks the modulating agent, a greater or lesser amount of each second label associated with each first label in the detection product, indicating that the modulating agent potentiates or attenuates binding between the first member, indicated by the first specific label, and the second member, indicated by the second specific label, in the presence of the third member. In one embodiment the method further comprises creating a modulating profile for each modulating agent, wherein the modulating profile indicates the relative modulation of binding between each first member and each second member of the binding pairs in the presence of the third member.

By “conditions that allow formation of detection products” means conditions in which first members can bind to second members, and preferably conditions in which modulating agents can bind to either the first member, second member, third member, or some combination thereof. Such conditions do not require that all first and second members bind or that all third members bind, only that the conditions allow for specific binding interactions to occur. These conditions include, for example, pH, time, temperature, and buffer composition, which allow binding between the members and agents of interest.

Where there is a third member of the binding complex and/or a third member of the detection product, the third member of the binding complex can potentiate or attenuate binding between the first member and second member of the binding complex. Thus, the third member can itself be a modulator (e.g., an agonist or antagonist), whose effect is modulated by the modulating agent. The third members and modulating agents are selected from a group consisting of cofactors, receptors, receptor ligands, proteins, peptides, protein domains, oligonucleotides, transcription factors, nucleic acids, small molecules, and small compounds, any of which can be synthetic, modified, or naturally occurring. In one embodiment, the first members are cofactors, the second member or members are receptors, and the third member is a receptor ligand. The modulators could result in formation of binding complexes that would not form in the absence of the modulating agent. Furthermore, a modulating agent can bind to the binding pair, the binding complex or any member thereof.

The invention further provides a method of screening, in a single assay, for an agent or agents with a selected binding profile, comprising (a) contacting the agent or agents to be screened with two or more putative binding members under conditions that allow the agent or agents to bind with the binding members to form detection products, wherein the binding members are coupled directly or indirectly to a set of microspheres labeled with a set of first labels, wherein each first label is specific for one binding member, wherein each agent to be screened is coupled directly or indirectly to a second label, and wherein each second label is specific for one agent to be screened; (b)detecting the presence of the first and second labels in the same detection products; (c) comparing the relative amount of each second label associated with each first label in the detection products, wherein a greater or lesser amount of each second label associated with each first label in the detection product indicates an agent, indicated by the second specific label, having greater or lesser binding to the binding members, indicated by the first specific label; (d) creating a binding profile for each agent to be screened, wherein the binding profile indicates the relative preference of binding between each agent to be screened and each binding member; and (e) comparing the binding profile for the agent or agents to be screened with the selected profile, the same binding profile indicating an agent with the selected binding profile. The binding members can be selected from a group consisting of cofactors, receptors, receptor ligands, proteins, peptides, protein domains, oligonucleotides, transcription factors, nucleic acids, small molecules, and small compounds. The agent or agents to be screened are selected from a group consisting of cofactors, receptors, receptor ligands, proteins, peptides, protein domains, oligonucleotides, transcription factors, nucleic acids, small molecules, and small compounds. In one embodiment, the binding members are cofactors and the agent or agents to be screened are receptors. For example, the method could be used to screen for orphan receptors or nuclear receptors having a selected binding profile.

By “putative binding members” is meant binding members that may or may not bind to the agent or agents to be screened to form a binding pair.

The method also provides a method of screening, in single assay, for an agent that modulates binding between two or more first members of a binding pair and one or more second members of a binding pair, comprising (a) contacting two or more first members and one or more second members of the binding pairs and the modulating agent, under conditions that allow formation of binding pairs; wherein the first members are coupled directly or indirectly to a set of microspheres labeled with a set of first labels; wherein each first label is specific for one first member; wherein the second member is coupled directly or indirectly to a second label; and wherein the second label is specific for one second member; (b) detecting the presence of the first specific labels and the second specific labels in a plurality of detection products, wherein each detection product comprises one microsphere; (c) comparing, to a control, the relative amount of each second label associated with each first label in the detection products, wherein the control lacks the agent to be screened, a greater or lesser amount of each second label associated with each first label in the detection products, indicating an agent that modulates binding between the specific first member, identified by the specific first label, and the specific second member, identified by the specific second label. Optionally, the method can further comprise creating a modulating profile for each agent to be screened, wherein the modulating profile indicates the relative modulation of binding between each first member and each second member of the binding pairs, and comparing the modulating profile for the agent to be screened to a selected profile. The agent to be screened can be selected from a group consisting of cofactors, receptors, receptor ligands, proteins, peptides, protein domains, oligonucleotides, transcription factors, nucleic acids, small molecules, and small compounds. In one embodiment, the first members are cofactors, the second member or members are receptors (including, for example, orphan or nuclear receptors), and the modulating agent is a small molecule or receptor ligand.

The invention also provides a method of screening, in a single assay, for an agent that modulates binding between the members of two or more binding complexes, comprising (a) contacting two or more first members, one or more second members, and a third member of the binding complexes with the agent to be screened, under conditions that allow formation of binding complexes, wherein each binding complex comprises one first and one second member; wherein the first members are coupled directly or indirectly to a set of microspheres labeled with a set of first labels; wherein each first label is specific for one first member; wherein the second member is coupled directly or indirectly to a second label; and wherein the second label is specific for one second member; (b) detecting the presence of the first specific labels and the presence or absence of the second specific labels in a plurality of detection products, wherein each detection product comprises one microsphere; (c) comparing, to a control, the relative amount of each second label associated with each first label in the detection products, wherein the control lacks the agent to be screened, a greater or lesser amount of each second label associated with each first label in the detection products, indicating an agent that modulates binding between the specific first member, identified by the specific first label, and the specific second member, identified by the specific second label, in the presence of the third member. Optionally, the screening method can further comprise creating a modulating profile for each agent to be screened, wherein the modulating profile indicates the relative modulation of binding between each first member and each second member of the binding pairs in the presence of the third member, and comparing the modulating profile for the agent to be screened to a selected profile.

The detection product can further comprise the third member of the binding complex and/or the modulating agent. The third member of the binding complex can potentiate or attenuate binding between the first member and second member of the binding complex. Thus, the third member may be a modulator, and the modulating agent used in this screening method may modulate the attenuation or potentiation caused by the third member. The agent to be screened can be selected from a group consisting of cofactors, receptors, receptor ligands, proteins, peptides, protein domains, oligonucleotides, transcription factors, nucleic acids, small molecules, and small compounds. In one embodiment, the first members are cofactors, the second member or members are receptors and the third member is a small molecule or receptor ligand and the modulating agent is a small molecule.

The invention also provides a set of microspheres coupled with a set of cofactors, wherein the microspheres are labeled with a label specific for each cofactor of the set. Specifically, the cofactors coupled to the microspheres can comprise nuclear receptor cofactors, and more specifically coactivators, corepressors, or both coactivators and corepressors. Even more specifically, the set of microspheres can be coupled to (1) coactivators comprising a five amino acid motif having, from amino to carboxy end, a leucine residue, two additional amino acid residues of any identity, and two leucine residues, (2) corepressors comprising a five amino acid motif having, from amino to carboxy end, a leucine residue, two additional amino acid residues, and two isoleucine residues, or (3) a combination thereof. The coactivators and corepressors coupled to the microspheres, more specifically, can comprise peptides having SEQ ID NO:1-43 or any subset thereof, including, for example, SEQ ID NO:1-5, SEQ ID NO:6-10, SEQ ID NO:11-15, SEQ ID NO:16-20, SEQ ID NO:21-25, SEQ ID NO:26-30, SEQ ID NO:31-35, SEQ ID NO:36-40, SEQ ID NO:41-43.

A “set of microspheres” comprises one or more subsets of microspheres wherein each subset comprises a plurality of microspheres labeled with the same label or combination of labels and wherein each label or combination of labels is specific for that subset.

Also, provided are kits for performing the methods of the present invention. For example, the invention provides a kit for confirming results of a phage display that identifies the amino acid sequences of two or more agents that bind to one or more specific binding members, comprising: (a) two or more subsets of microspheres having specific first labels, wherein each microsphere having a specific label is separated from microspheres having a different specific label; (b) a means for coupling each set of microspheres selectively to one agent; and (c) the specific binding member or members and a label or labels for coupling directly or indirectly to a specific binding member; or (c′) the specific binding member or members, wherein each ligand is coupled directly or indirectly to a second label that is specific for one ligand. The invention also provides a kit for confirming the results of a phage display that identifies amino acid sequences of two or more agents that modulate binding between one or more selected binding pairs or complexes.

The present invention is more particularly described in the following examples which are intended as illustrative only since numerous modifications and variations therein will be apparent to those skilled in the art.

Throughout this application, various publications are referenced. The disclosures of these publications in their entireties are hereby incorporated by reference into this application in order to more fully describe the state of the art to which this invention pertains.

Although the present process has been described with reference to specific details of certain embodiments thereof, it is not intended that such details should be regarded as limitations upon the scope of the invention except as and to the extent that they are included in the accompanying claims.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### EXAMPLES

### Example 1: Coupling of Biotinylated Peptides to the Microspheres

Peptide sequences with biotin at the amino terminus followed by a 17-27 amino acid sequence containing either an LXXLL or an LXXII-motif sequence were synthesized by Synpep Corporation (Dublin, Calif.). Each peptide was purified by reverse-phase HPLC. Amino acid composition and peptide concentration were determined by conventional methods for each peptide sequence. Each biotinylated peptide shown in Table 1 was coupled to a specific set of microspheres with a unique fluorescent profile. Lumavidin-coated polystyrene microsphere sets (5.5 mm in diameter) with different ratios of red and orange fluorescence were purchased from the Luminex Corporation (Austin, Tex.). The Lumavidin-coated microspheres (200,000) were added to 400 μl buffer (phosphate buffered saline containing 0.02% Tween-20, 0.1% bovine serum albumin, 0.02% sodium azide and 1 mM dithiothreitol). The microspheres were centrifuged and resuspended in 400 μl fresh buffer and were then incubated with 500 ng biotinylated peptide for 30 min at room temperature in the dark. The microsphere suspension was washed twice and resuspended in 0.1 ml buffer. Free D-biotin (50 μl of 5 mM D-biotin) was added to each microsphere set and was incubated for 30 min at room temperature in the dark. The microspheres were washed twice in 0.25 ml buffer and resuspended in 0.2 ml buffer.

TABLE 1

| | | |
|---|---|---|
| SRC-1 (626-642) | B-YSQTSHKLVQLLTTTAE-COOH | (SEQ ID NO:1) |
| SRC-1 (LCD2, 676-700) | B-CPSSHSSLTERHKILHRLLQEGSPS-CONH2 | (SEQ ID NO:2) |
| SRC-1 (LCD3, 735-759) | B-LDASKKKESKDHQLLRYLLDKDEKD-CONH2 | (SEQ ID NO:3) |
| CBP (LCD1, 58-80) | B-NLVPDAASKHKQLSELLRGGSGS-CONH2 | (SEQ ID NO:4) |
| CBP (LCD2, 346-368) | B-ADPEKRKLIQQQLVLLLHAHKCQ-CONH2 | (SEQ ID NO:5) |
| CBP (LCD3, 2055-2078) | B-SVQPPRSISPSALQDLLRTLKSP-CONH2 | (SEQ ID NO:6) |
| TRIP2 (54-80) | B-PVSSMAGNTKNHPMLMNLLKDNPAQDF-CONH2 | (SEQ ID NO:7) |
| TIF2 (676-702) | B-GSTHGTSLKEKHKILHRLLQDSSSPVD-CONH2 | (SEQ ID NO:8) |
| RAC3 (671-697)¹ | B-SNMHGSLLQEKHRILHKLLQNGNSPAE-CONH2 | (SEQ ID NO:9) |
| RIP140 (805-831) | B-PVSPQDFSFSKNGLLSRLLRQNQDSYL-CONH2 | (SEQ ID NO:10) |
| TRIP9 (275-301) | B-RTPLGSAMLRPNPILARLLRAHGAPEP-CONH2 | (SEQ ID NO:11) |
| P300 (1) (67-93) | B-SLGMVQDAASKHKQLSELLRSGSSPNL-CONH2 | (SEQ ID NO:12) |
| AIB1 (607-631), SRC-3, | B-ENQRGPLESKGHKKILLQLLTCSSDD-CONH2 | (SEQ ID NO:13) |
| ACTR, RAC3) | | |
| TIF2 (627-651) | B-DGQSRLHDSKGQTKLLQLLTTKSDQ-CONH2 | (SEQ ID NO:14) |
| RIP140 (922-946) | B-EHRSWARESKSFNVLKQLLLSENCV-CONH2 | (SEQ ID NO:15) |
| SRC-1 (32-56) | B-EKRRREQEWKYLEELAELLSANISD-CONH2 | (SEQ ID NO:16) |
| TIF2 (864-888) | B-RISQSTFNNPRPGQLGRLLPNQNLP-CONH2 | (SEQ ID NO:17) |
| TRIP8 (26-47)² | B-KKKLVSQNNEQGSTLRDLLTTTAGK-CONH2 | (SEQ ID NO:18) |
| p300/AF (176-200) | B-HKEEDADTKQVYFYLFKLLRKSILQ-CONH2 | (SEQ ID NO:19) |
| TIF1 (374-394) | B-STALLYSKRLITYRLRHLLRARCDA-CONH2 | (SEQ ID NO:20) |
| P300 (2037-2061) | B-GISPLKPGTVSQQALQNLLRTLRSP-CONH2 | (SEQ ID NO:21) |
| Pcip (1027-1051)³ | B-GPHGSQNRPLLRNSLDDLLGPPSNA-CONH2 | (SEQ ID NO:22) |
| AIB1 (102-123, Pcip, SRC-3, | B-KKKSTGQGVIDKDSLGPLLLQALDG-CONH2 | (SEQ ID NO:23) |
| AIB1, ACTR, RAC3)^{2,4} | | |
| RIP140 (10-31)² | B-KKKDVHQDSIVLTYLEGLLMHQAAG-CONH2 | (SEQ ID NO:24) |
| TIF1 (711-732)² | B-KKKRPQNANYPRSILTSLLLNSSQS-CONH2 | (SEQ ID NO:25) |
| TRIP4 (23-47) | B-TKFVNLYTRERQDRLAVLLPGRHPC-CONH2 | (SEQ ID NO:26) |
| ACTR (1041-1062, SRC-3, | B-KKKGQSDERALLDQLHTLLSNTDAT-CONH2 | (SEQ ID NO:27) |
| RAC3, AIB1))^{2,5} | | |
| ACTR (734-758, RAC3, | B-QEQLSPKKKENNALLRYLLDRDDPS-CONH2 | (SEQ ID NO:28) |
| SRC-3, AIB1, pCIP)⁶ | | |
| TIF2 (732-756) | B-QEPVSPKKKENALLRYLLDKDDTKD-CONH2 | (SEQ ID NO:29) |
| TRIP3 (85-109) | B-SLQNLKNLGESATLRSLLLNPHLRQ-CONH2 | (SEQ ID NO:30) |
| RIP14O (487-511) | B-SKNSKLNSHQKVTLLQLLLGHKNEE-CONH2 | (SEQ ID NO:31) |
| RIP140 (699-723) | B-SGSEIENLLERRTVLQLLLGNPTKG-CONH2 | (SEQ ID NO:32) |
| RIP140 (366-390) | B-LERNNIKQAANNSLLLHLLKSQTIP-CONH2 | (SEQ ID NO:33) |
| RIP14O (119-143) | B-MVDSVRKGKQDSTLLASLLQSFSSR-CONH2 | (SEQ ID NO:34) |
| RIP140 (172-196) | B-EKDLRCYGVASSHLKTLLKKSKVKD-CONH2 | (SEQ ID NO:35) |
| TRIP9 (61-85) | B-ALHLAVIHQHEPFLDFLLGFSAGTE-CONH2 | (SEQ ID NO:36) |
| TRIPS (14-38) | B-EQNVSSLNEREQELHNLLEVVSQCC-CONH2 | (SEQ ID NO:37) |
| NCOR (2040-2065) | B-GQVPRTHRLITLADHICQIITQDFAR-CONH2 | (SEQ ID NO:38) |
| NCOR (2251-2275) | B-GHSFADPASNLGLEDIIRKALMGSF-CONH2 | (SEQ ID NO:39) |
| SMRT (2124-2149) | B-PGVKGHQRVVTLAQHISEVITQDYTR-CONH2 | (SEQ ID NO:40) |
| SMRT (2329-2354) | B-SQAVQEHASTNMGLEAIIRKALMGKY-CONH2 | (SEQ ID NO:41) |
| Repressor of ER Activity | B-PAGPRGMGTALKLLLGAGAVAYGVR-CONH2 | (SEQ ID NO:42) |
| (REA) (13-37) | | |
| Repressor of ER Activity | B-AIFFNRIGGVQQDTILAEGLHFRIP-CONH2 | (SEQ ID NO:43) |
| (REA) (49-73) | | |

¹Same sequence for ACTR (681-707) and AIB1 (671-697).
²KKK was added to native sequence to improve solubility.
³Mouse Sequence.
⁴Same sequence as SRC-3 (102-123), ACTR (617-641) and RAC 3 (607-631).
⁵Same sequence as SRC (1041-1062), RAC 3 (1046-1067), and Aib1 (1046-1067)
⁶Same sequence as RAC 3 (734-758), SRC-3 (734-758), Aib1 (724-748), and Pcip (716-740).

### Example 2: Determination of Peptide Coupling Efficiency

Peptide coupling efficiency was determined by quantifying the biotin-binding capacity of the microsphere sets both before and after peptide coupling. Aliquots of microspheres from above (3,000) in 0.1 ml were incubated with an excess amount of biotin-FITC (5 μl of 1 mM biotin-FITC) (Molecular Probes, Eugene, Oreg.) for 30 min at room temperature in the dark. The microspheres were washed twice with buffer, then blocked with free biotin as described above. The FITC fluorescence-associated with each microsphere set was measured by flow cytometry (FACSCalibur, Becton Dickinson, San Jose, Calif.) and quantified using Quantum Fluorescence Kit for MESF units of FITC calibration particles (SIGMA, St Louis, Mo.).

### Example 3: Expression of Human Estrogen Receptor β Ligand Binding Domain (ERβ LBD)

Human Estrogen Receptor β Ligand Binding Domain (ERβ LBD) was expressed in E.coli strain BL21(DE3) as an amino-terminal poly-histidine tagged fusion protein. Expression was under the control of an IPTG inducible T7 promoter. DNA encoding this recombinant protein was subcloned into the pRSET-A expression vector (Invitrogen, Carlsbad, Calif.). The encoded sequence of the polyhistidine tag (MKKGHHHHG) (SEQ ID NO:44) was incorporated into the 5′ PCR amplification primer, which was upstream of DNA encoding residues 250-530 of ERβ. The coding sequence of ERβ LBD was derived from GenBank Accession Number AF051427.

Ten-liter fermentation batches were grown in LB media with 0.1 mg/ml Ampicillin at 22° C. for 16 hours until OD600=14. At this cell density, 0.25 mM IPTG was added and induction proceeded for 4 hours to a final OD600=16. Cells were harvested by centrifugation (20 minutes, 3500 g, 4° C.), and concentrated cell slurries were stored in PBS at −80° C.

### Example 4: Purification of ERβ LBD

30-40 g cell paste (equivalent to 2-3 liters of the fermentation batch) was resuspended in 300-400 mL TBS, pH 8.0(25 mM Tris, 150 mM NaCl). Cells were lysed by passing 3 times through a homogenizer (Rannie, Copenhagen, Denmark), and cell debris was removed by centrifugation (30 minutes, 20,000 g, 4° C.). The cleared supernatant was filtered through coarse pre-filters, and TBS pH 8.0 containing 500 mM imidazole was added to obtain a final imidazole concentration of 50 mM. This lysate was loaded onto a column (6×8 cm) packed with Sepharose [Ni⁺⁺ charged] Chelation resin (Pharmacia, Piscataway, N.J.) and pre-equilibrated with TBS pH 8.0/ 50 mM imidazole. After washing to baseline absorbance with equilibration buffer, the column was developed with a linear gradient of 50 to 365 mM Imidazole in TBS pH 8.0. Column fractions were pooled and dialyzed against TBS pH 8.0 containing 5% 1,2-propanediol, 5 mM DTT and 0.5 mM EDTA. The protein sample was concentrated using Centri-prep 10K (Amicon, Waters, Millipore, Bedford, Mass.) and subjected to size exclusion chromatography using a column (3×90 cm) packed with Sepharose S-75 resin (Pharmacia) pre-equilibrated with TBS pH 8.0 containing 5% 1,2-propanediol, 5 mM DTT and 0.5 mM EDTA.

### Example 5: Biotinylation of ERβ LBD

Purified ERβ LBD was buffer exchanged using PD-10 gel filtration columns (Pharmacia) into PBS [100 mM NaPhosphate, pH 7.2, 150 mM NaCl]. ERβ LBD was diluted to approximately 30 μM in PBS and five-fold molar excess of NHS-LC-Biotin (Pierce, Rockford, Ill.) was added in a minimal volume of PBS. This solution was incubated with gentle mixing for 60 minutes at ambient room temperature. The biotinylation modification reaction was stopped by the addition of 2000×-molar excess of Tris-HCl pH 8.0. The modified ERβ LBD was dialyzed against 2 buffer changes, each of at least 50 volumes; TBS pH 8.0 containing 5 mM DTT, 2 mM EDTA and 2% sucrose. This modified protein was distributed into aliquots, frozen on dry ice and stored at −80C. The biotinylated ERβ LBD was subjected to mass spectrometric analysis to reveal the extent of modification by the biotinylation reagent. In general, approximately 95% of the protein had at least a single site of biotinylation; and the overall extent of biotinylation followed a normal distribution of multiple sites, ranging from one to seven.

### Example 6: Coupling of ERβ LBD to Fluorochrome

Purified and biotinylated recombinant ERβ LBD was coupled to the green fluorochrome Alexa 488 by incubating with NeutravidinAlexa 488(Molecular Probes, Eugene, Oreg.) at a 10:1 molar ratio for 2 min at room temperature. The coupling reaction was stopped by the addition of 30 μl free D-biotin (5 mM). The receptor concentration was adjusted in buffer prior to addition to the microsphere suspension.

### Example 7: Preparation of Agents or Ligands

All agents and ligands (i.e., estradiol (Sigma Chemical Co., St. Louis, Mo.), Agents 1-6, and GI 165638) were dissolved in DMSO at 10 mM and then diluted with buffer to the appropriate concentration prior to addition to the microsphere suspension.

### Example 8: Multiplexed Binding Experiment to Determine Functional Molecular Interactions

The agents and ligands were prepared according to Example 7. All components (3,500 microspheres of each subset, +/−agent(s), +/−receptor:Alexa 488) were added in a total volume of 0.35 ml buffer. The suspension incubated for 1.5 to 2 hours at room temperature in the dark. The fluorescence associated with each microsphere was measured by flow cytometry.

### Example 9: Flow Cytometric Analysis

Microsphere fluorescence was measured using a FACSCalibur flow cytometer (Becton Dickinson, San Jose, Calif.) equipped with Luminex Lab MAP hardware and software (Luminex Corp., Austin, Tex.). All green fluorescence measurements were converted to molecules of equivalent soluble fluorochrome (MESF) using Quantum Fluorescence Kit for MESF units of FITC calibration particles and QuickCal software (all obtained from Sigma, St. Louis, Mo.). Green fluorescence contributed by the microspheres alone has been subtracted from all data points.

### Example 10: Binding Profiles of ERβ LBD and PPARγ LBD

Four subsets of microspheres (each subset was coupled to either no peptide, SRC-1(2)(676-700), SRC-1(1)(626-642), or CBP-1(58-80) peptide as described above) were incubated for approximately 1.5 hours with either no receptor, ERβ LBD or PPARγ LBD at 1 nM. ERβ LBD and PPARγ LBD were expressed, purified, biotinylated, and coupled to fluorochrome as described above. The fluorescence associated with each microsphere was determined by flow cytometric analysis. The results are shown in FIG. 1.

### Example 11: ERβ LBD BGinding to 34 LXXLL-motif Coactivator Peptides in the Presence or Absence of Estradiol

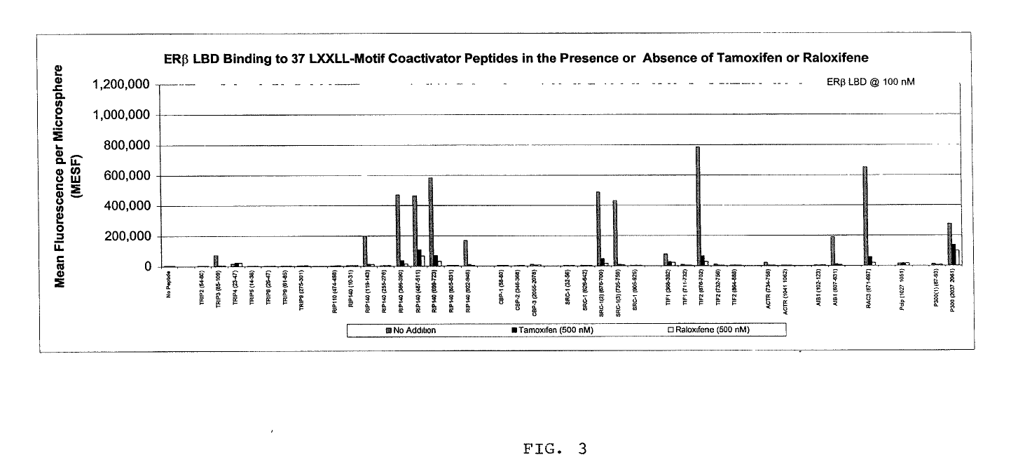

Thirty eight microsphere subsets (each subset was coupled to either no peptide, or to one of 34 different coactivator peptides as described above) were incubated with ERβ LBD at 100 nM (ERβ LBD was expressed, purified, biotinylated, and coupled to fluorochrome as described above) in the presence or absence of estradiol (1 mM) for approximately 1.5 hours. The fluorescence associated with each microsphere as determined by flow cytometric analysis. The results are shown in FIG. 2.

### Example 12: ERβ LBD Binding to 37 Different Coactivator Peptides: Effect of Tamoxifen, and Raloxifene

Thirty-nine microsphere subsets (each subset was coupled to either no peptide or to one of 37 different coactivator peptides as described above) were incubated with ERβ LBD at 100 nM (ERβ LBD was expressed, purified, biotinylated, and coupled to fluorochrome as described above) in the presence or absence of tamoxifen(500 nM) or raloxifene (500 nM) for approximately 1.5 hours. The fluorescence associated with each microsphere was determined by flow cytometric analysis. The data are shown in FIG. 3.

### Example 13: Binding Profile of LBDs Derived from Recptor B, Receptor C and PPAR γ LBD for Cofactor Peptides

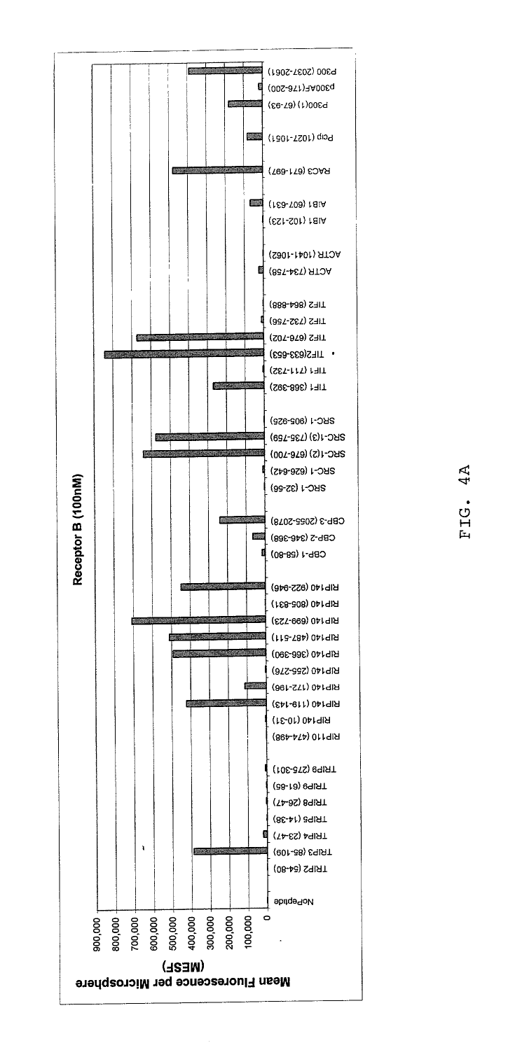

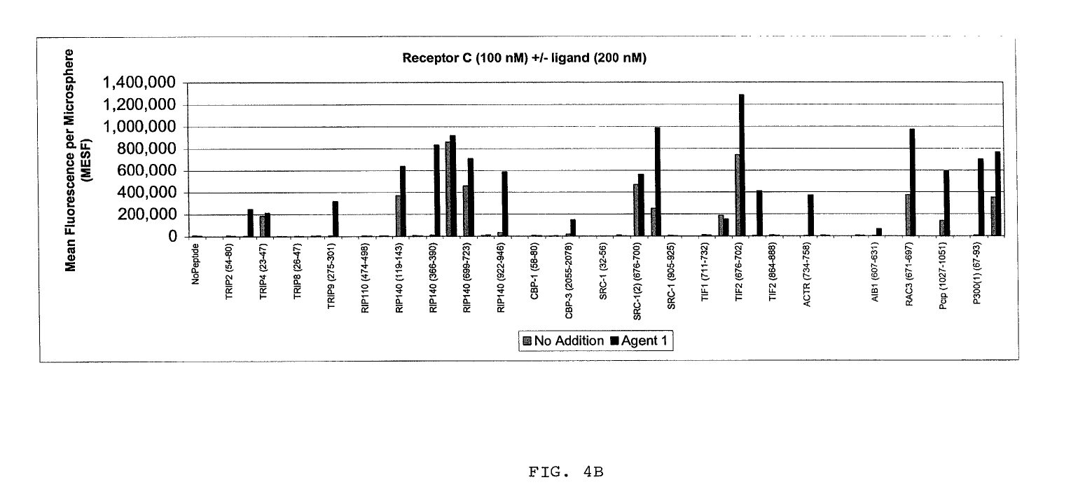

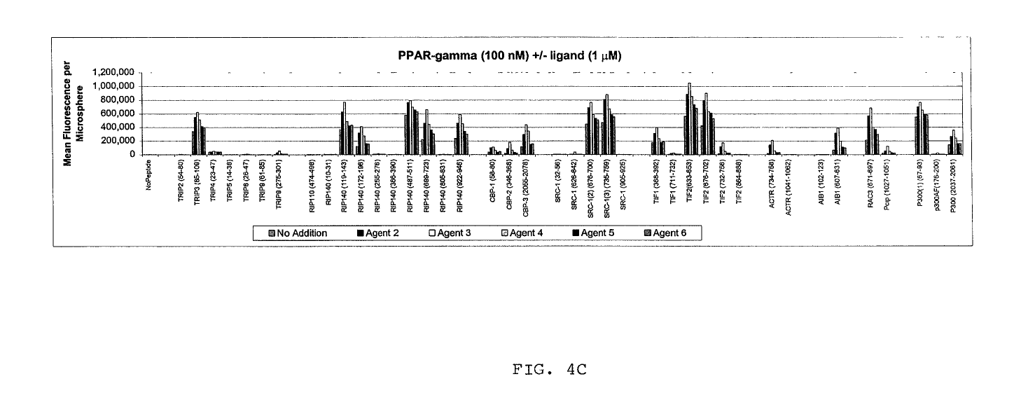

Microsphere subsets (each subset was coupled to either no peptide, or to one of 40 different coactivator peptides described above) were incubated with PPAR γ LBD or Receptor B or C at 100 nM (receptors were expressed, purified, biotinylated, and coupled to fluorochrome as described above) in the presence or absence of agent (0.2 or 1 μM) for approximately 1.5 hours. The fluorescence associated with each microsphere was determined by flow cytometric analysis. The data are shown in FIG. 4A-C.

### Example 14: ERβ LBD Binding to Coactivator Peptides (src-1) and Corepressor peptide (SMRT) in the Presence or Absence of Estradiol

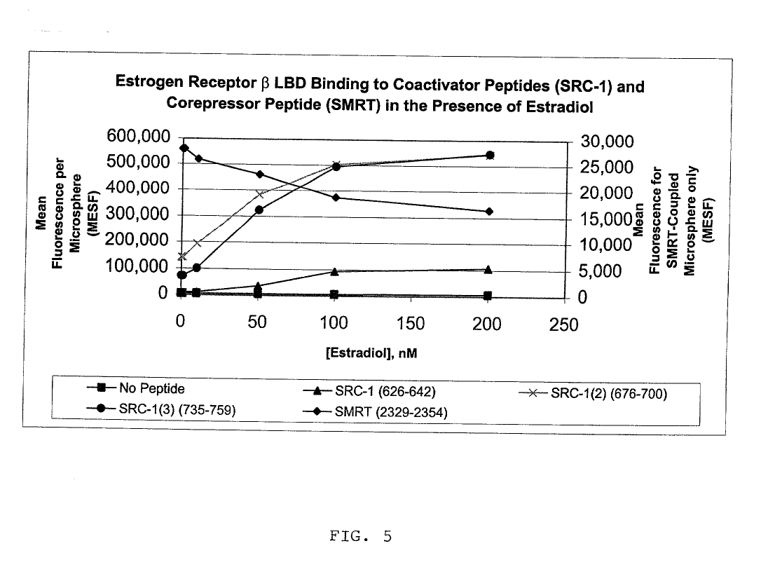

Microsphere subsets (coupled to either no peptide, SRC-1(3)(735-759), SRC-1(626-642), SRC-1(2)(676-700) or SMRT (2329-2354) as described above)were incubated with ERβ LBD at 100 nM (expressed, purified, biotinylated, and coupled to fluorochrome as described above) in the presence of estradiol, at the indicated concentration, for approximately 1.5 hours. The fluorescence associated with each microsphere was determined by flow cytometric analysis. The data are shown in FIG. 5.

### Example 15: Effect of Tamoxifen, Raloxifene and GI 165638 on Estradiol-enhanced ERβ LBD Binding to Cofactor Peptides

Microsphere subsets (coupled to cofactors as described above) are incubated with ERβ LBD at 100 nM (expressed, purified, biotinylated, and coupled to fluorochrome as described above) in the presence of estradiol, and raloxifene, or tamoxifen, for approximately 1.5 hours. The fluorescence associated with each microsphere is determined by flow cytometric analysis.

### Example 16: Effect of Estradiol, Raloxifene or Tamoxifen on ER α LBD Binding to Cofactor Peptides

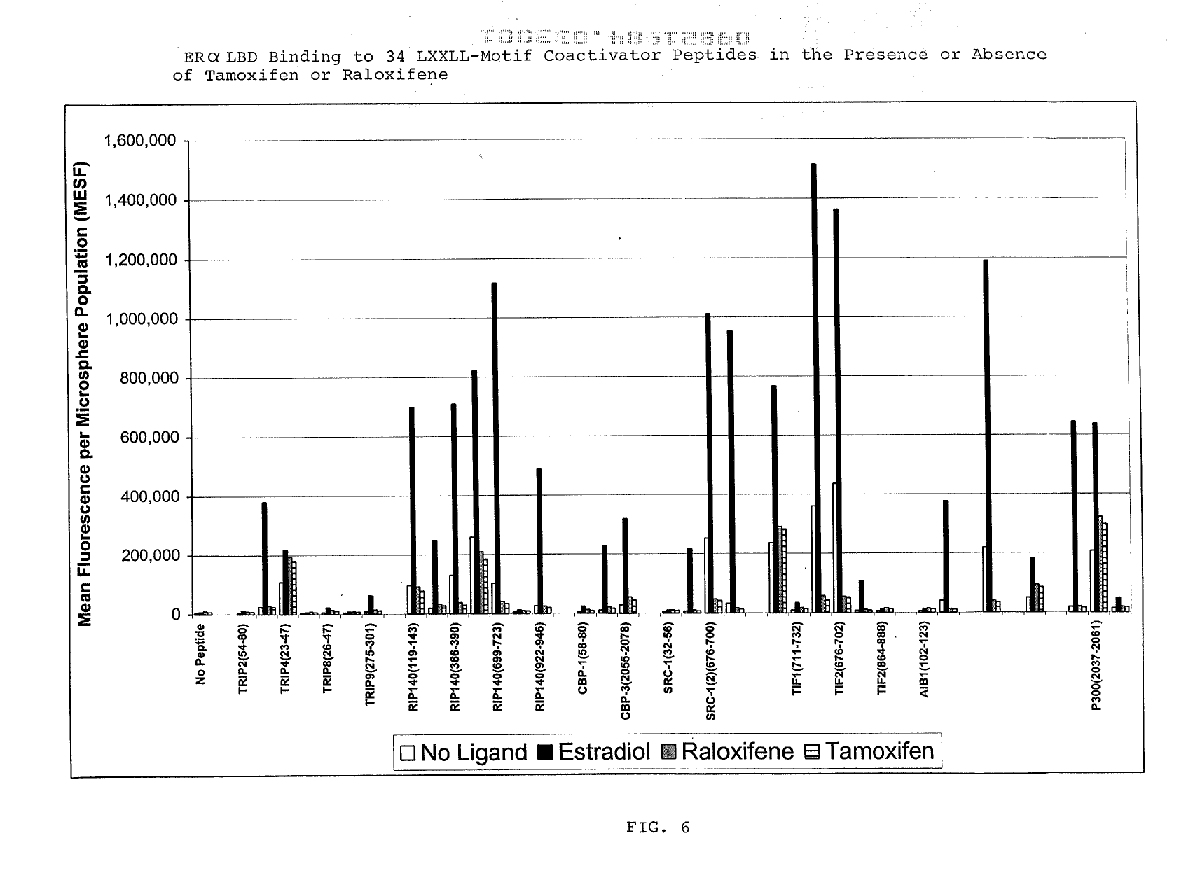

Thirty-four fluorescently distinct red-orange Lumavidin-coated microsphere populations (Luminex Corp., Austin, Tex.) were separately coupled to 34 biotin-containing peptide sequences and combined. A separate microsphere population was also included that had no peptide coupled to its surface. Each 17-27 amino acid sequence represented a specific coactivator protein containing one unique LXXLL motif as described above. Each coactivator peptide designation on the x-axis of the graph in FIG. 6 includes GenBank specified amino acid residues. Biotinylated recombinant ER α LBD was coupled to Alexa 488 Streptavidin and incubated with the multiplexed set of coactivator peptide-coupled microspheres in the presence or absence of ligand. The green fluorescence associated with each microsphere was analyzed after 1.5 hours of incubation on a FACSCalibur flow cytometer (Becton Dickinson, San Jose, Calif.) equipped with Luminex LabMAP hardware and software. All background fluorescence contributed by the microspheres alone has been subtracted. FIG. 6 shows ER α LBD (100 nM) binding to coactivator peptide-coupled microspheres either in the absence of ligand, or in the presence of estradiol (2 μM), raloxifene (500 nM) or tamoxifen (500 nM). This entire experiment was conducted in 4 tubes. Each tube contained 35 distinct microsphere populations. Estrogen enhanced the ER α LBD binding to coactivator peptides while the antiestrogenic compounds raloxifene and tamoxifen, generally inhibited ER α LBD binding to coactivator peptides. The data showed the binding preference of ER α LBD for certain coactivators such as steroid receptor coactivator-1(SRC-1), transcriptional intermediary factor 2(TIF-2) and RIP140 over cAMP response element binding protein (CBP).

### Example 17: Classification of Compound Activities Based on Binding Patterns to ER α LBD Conformation-sensing Peptides

Microsphere populations are coupled to synthetic biotinylated conformation-sensing peptide sequences using coupling techniques described above. Nuclear receptor modulators appear to induce very specific conformational changes in nuclear receptor physical structure. Conformation-sensing peptide sequences preferentially bind to a given nuclear receptor (or may decrease binding) upon exposure to ligands that result in certain conformational changes in the nuclear receptor. These peptide sequences may be identified by affinity-selection phage display according to the techniques described by Chang et al. (1999) Dissection of the LXXLL nuclear receptor-coactivator interaction motif using combinatorial peptide libraries: discovery of peptide antagonists of estrogen receptors alpha and beta, Mol. Cell Biol. 19:8226-8239; by Norris et al (1999) Peptide antagonists of the human estrogen receptor. Science 285:744-746; and by Paige et al. (1999) Estrogen receptor (ER) modulators each induce distinct conformational changes in ER alpha and ER beta. P.N.A.S. USA 96:3999-4004, which are each incorporated herein by reference for their affinity-selection phage display techniques. The multiplexed peptide-coupled microsphere populations are incubated with nuclear receptor (e.g. ER α LBD, 10 nM) in the presence or absence of compound (1 mM) for approximately 1.5 hours. Based on the binding profile of ER α LBD to the peptide set, and comparison to known control ligands, one may rapidly classify and/or screen for compounds that stabilize specific conformational structures. The advantage of the multiplexed screening approach is that one may characterize a potential nuclear receptor modulator for many conformational states in a single assay volume.

What is claimed is:

1. A method of identifying, in a single assay, the relative binding of two or more first members of a binding pair to one or more second members of a binding pair, comprising
(a) contacting the first members with the second member or members under conditions that allow the first members to bind with the second member or members to form binding pairs, wherein the first members are coupled directly or indirectly to a set of microspheres labeled with a set of first labels, wherein each first label is specific for one first member, wherein each second member is coupled directly or indirectly to a second label, and wherein each second label is specific for one second member;
(b) detecting the presence of the first label and the presence or absence of the second label in a plurality of detection products, wherein each detection product comprises one microsphere;
(c) comparing the relative amount of each second label associated with each first label in the detection products, wherein a greater or lesser amount of each second label associated with each first label in the detection product indicates first members, identified by the specific first label, having greater or lesser binding to the second member or members, indicated by the second specific label; and
(d) creating a binding profile for each second member,
the binding profile indicating the relative preference of binding between each first member and each second member of the binding pairs.

2. The method of claim 1, wherein the first and second labels are radiolabels, dyes, fluorescent labels, or a combination thereof.

3. The method of claim 1, wherein the microspheres contain the first labels.

4. The method of claim 1, wherein one or more detection products further comprise a second label.

5. The method of claim 1, wherein the microspheres are strepavidin-coated and wherein the first members are biotinylated.

6. The method of claim 1, wherein the microspheres are carboxylated and wherein the first members each have one or more free amine groups.

7. The method of claim 1, wherein the microspheres are maleimide-coated and wherein the first members each have one or more sulfhydryl reactive entities.

8. The method of claim 1, wherein the first and second labels are detected and distinguished with a detection device with a light source, wherein the detection device is capable of detecting and distinguishing each detection product and each label in the same detection product.

9. The method of claim 8, wherein the first and second labels are detected and distinguished by passing the microspheres across or through the light.

10. The method of claim 8, wherein the first and second labels are detected and distinguished by passing the light across or through the microspheres.

11. The method of claim 8, wherein microspheres are on a two-dimensional surface.

12. The method of claim 1, wherein the first members are selected from a group consisting of cofactors, receptors, receptor ligands, proteins, peptides, protein domains, oligonucleotides, transcription factors, nucleic acids, small molecules, and small compounds.

13. The method of claim 1, wherein the second members are selected from a group consisting of cofactors, receptors, receptor ligands, proteins, peptides, protein domains, oligonucleotides, transcription factors, nucleic acids, small molecules, and small compounds.

14. The method of claim 1, wherein the first members are cofactors and the second member or members are receptors.

15. The method of claim 14, wherein the cofactors are coactivators, corepressors, or a combination of coactivators and corepressors.

16. The method of claim 14, wherein the receptor or receptors are orphan receptors.

17. The method of claim 14, wherein the receptor or receptors are nuclear receptors.

18. A method of identifying, in a single assay, the relative modulation, by a modulating agent, of binding between two or more binding pairs, comprising
(a) contacting two or more first members and one or more second members of the binding pairs and the modulating agent, under conditions that allow formation of binding pairs, wherein each binding pair comprises one first and one second member of the binding pair; wherein the first members are coupled directly or indirectly to a set of microspheres labeled with a set of first labels; wherein each first label is specific for one first member; wherein the second member is coupled directly or indirectly to a second label; and wherein the second label is specific for one second member;
(b) detecting the presence of the first specific labels and the presence or absence of the second specific labels in a plurality of detection products, wherein each detection product comprises one microsphere; and
(c) comparing, to a control, the relative amount of each second label associated with each first label in the detection products, wherein the control lacks the modulating agent;
a greater or lesser amount of each second label associated with each first label in the detection product, indicating that the modulating agent potentiates or attenuates binding between the first member, indicated by the first specific label, and the second member, indicated by the second specific label.

19. The method of claim 18, further comprising creating a modulating profile for each modulating agent, wherein the modulating profile indicates the relative modulation of binding between each first member and each second member of the binding pairs.

20. The method of claim 18, wherein the first and second labels are radiolabels, dyes, fluorescent labels, or a combination thereof.

21. The method of claim 18, wherein the microspheres contain the first labels.

22. The method of claim 18, wherein one or more detection products further comprise a second label.

23. The method of claim 18, wherein the microspheres are strepavidin-coated and wherein the first members are biotinylated.

24. The method of claim 18, wherein the microspheres are carboxylated and wherein the first members each have one or more free amine groups.

25. The method of claim 18, wherein the microspheres are maleimide-coated and wherein the first members each have one or more sulfhydryl reactive entities.

26. The method of claim 18, wherein the first and second labels are detected and distinguished with a detection device with a light source, wherein the detection device is capable of detecting and distinguishing each detection product and each label in the same detection product.

27. The method of claim 26, wherein the first and second labels are detected and distinguished by passing the microspheres across or through the light.

28. The method of claim 26, wherein the first and second labels are detected and distinguished by passing the light across or through the microspheres.

29. The method of claim 26, wherein microspheres are on a two-dimensional surface.

30. The method of claim 18, wherein the first members are selected from a group consisting of cofactors, receptors, receptor ligands, proteins, peptides, protein domains, oligonucleotides, transcription factors, nucleic acids, small molecules, and small compounds.

31. The method of claim 18, wherein the second members are selected from a group consisting of cofactors, receptors, receptor ligands, proteins, peptides, protein domains, oligonucleotides, transcription factors, nucleic acids, small molecules, and small compounds.

32. The method of claim 18, wherein the modulating agent is selected from a group consisting of cofactors, receptors, receptor ligands, proteins, peptides, protein domains, oligonucleotides, transcription factors, nucleic acids, small molecules, and small compounds.

33. The method of claim 18, wherein the first members are cofactors, the second member or members are receptors, and the modulating agent is a receptor ligand.

34. The method of claim 33, wherein the cofactors are coactivators, corepressors, or a combination of coactivators and corepressors.

35. The method of claim 33, wherein the receptor or receptors are orphan receptors.

36. The method of claim 33, wherein the receptor or receptors are nuclear receptors.

37. A method of identifying, in a single assay, the relative modulation, by a modulating agent, of binding between the members of two or more binding complexes, comprising
(a) contacting two or more first members, one or more second members, a third member of the binding complexes, and the modulating agent, under conditions that allow formation of binding complexes, wherein each binding complex comprises one first and one second member of the binding complex; wherein the first members are coupled directly or indirectly to a set of microspheres labeled with a set of first labels; wherein each first label is specific for one first member; wherein the second member is coupled directly or indirectly to a second label; and wherein the second label is specific for one second member;
(b) detecting the presence of the first specific labels and the presence or absence of the second specific labels in a plurality of detection products, wherein each detection product comprises one microsphere;
(c) comparing, to a control, the relative amount of each second label associated with each first label in the detection products, wherein the control lacks the modulating agent
a greater or lesser amount of each second label associated with each first label in the detection product, indicating that the modulating agent potentiates or attenuates binding between the first member, indicated by the first specific label, and the second member, indicated by the second specific label, in the presence of the third member.

38. The method of claim 37, further comprising creating a modulating profile for each modulating agent, wherein the modulating profile indicates the relative modulation of binding between each first member and each second member of the binding pairs in the presence of the third member.

39. The method of claim 37, wherein one or more binding complex further comprises one third member of the binding complex.

40. The method of claim 37, wherein the third member of the binding complex potentiates or attenuates binding between the first member and second member of the binding complex.

41. The method of claim 37, wherein the first and second labels are radiolabels, dyes, fluorescent labels, or a combination thereof.

42. The method of claim 37, wherein the microspheres contain the first labels.

43. The method of claim 37, wherein one or more detection products further comprise a second label.

44. The method of claim 37, wherein the microspheres are strepavidin-coated and wherein the first members are biotinylated.

45. The method of claim 37, wherein the microspheres are carboxylated and wherein the first members each have one or more free amine groups.

46. The method of claim 37, wherein the microspheres are maleimide-coated and wherein the first members each have one or more sulfhydryl reactive entities.

47. The method of claim 37, wherein the first and second labels are detected and distinguished with a detection device with a light source, wherein the detection device is capable of detecting and distinguishing each detection product and each label in the same detection product.

48. The method of claim 47, wherein the first and second labels are detected and distinguished by passing the microspheres across or through the light.

49. The method of claim 47, wherein the first and second labels are detected and distinguished by passing the light across or through the microspheres.

50. The method of claim 47, wherein microspheres are on a two-dimensional surface.

51. The method of claim 37, wherein the first members are selected from a group consisting of cofactors, receptors, receptor ligands, proteins, peptides, protein domains, oligonucleotides, transcription factors, nucleic acids, small molecules, and small compounds.

52. The method of claim 37, wherein the second members are selected from a group consisting of cofactors, receptors, receptor ligands, proteins, peptides, protein domains, oligonucleotides, transcription factors, nucleic acids, small molecules, and small compounds.

53. The method of claim 37, wherein the modulating agent is selected from a group consisting of cofactors, receptors, receptor ligands, proteins, peptides, protein domains, oligonucleotides, transcription factors, nucleic acids, small molecules, and small compounds.

54. The method of claim 37, wherein the first members are cofactors, the second member or members are receptors, and the third member is a receptor ligand.

55. The method of claim 54, wherein the cofactors are coactivators, corepressors, or a combination of coactivators and corepressors.

56. The method of claim 54, wherein the receptor or receptors are orphan receptors.

57. The method of claim 54, wherein the receptor or receptors are nuclear receptors.

58. A method of screening, in a single assay, for an agent or agents with a selected binding profile, comprising
(a) contacting the agent or agents to be screened with two or more putative binding members under conditions that allow the agent or agents to bind with the binding members to form binding pairs, wherein the binding members are coupled directly or indirectly to a set of microspheres labeled with a set of first labels, wherein each first label is specific for one binding member, wherein each agent to be screened is coupled directly or indirectly to a second label, and wherein each second label is specific for one agent to be screened;
(b) detecting the presence of the first and second labels in a plurality of detection products, wherein each detection product comprises one microsphere;
(c) comparing the relative amount of each second label associated with each first label in the detection products, wherein a greater or lesser amount of each second label associated with each first label in the detection product indicates an agent, indicated by the second specific label, having greater or lesser binding to the binding members, indicated by the first specific label;
(d) creating a binding profile for each agent to be screened, wherein the binding profile indicates the relative preference of binding between each agent to be screened and each binding member; and
(e) comparing the binding profile for the agent or agents to be screened with the selected profile,
the same binding profile indicating an agent with the selected binding profile.

59. The method of claim 58, wherein the first and second labels are radiolabels, dyes, fluorescent labels, or a combination thereof.

60. The method of claim 58, wherein the microspheres contain the first labels.

61. The method of claim 58, wherein one or more detection products further comprise a second label.

62. The method of claim 58, wherein the microspheres are strepavidin-coated and wherein the binding members are biotinylated.

63. The method of claim 58, wherein the microspheres are carboxylated and wherein the binding members each have one or more free amine groups.

64. The method of claim 58, wherein the microspheres are maleimide-coated and wherein the binding members each have one or more sulfhydryl reactive entities.

65. The method of claim 58, wherein the first and second labels are detected and distinguished with a detection device with a light source, wherein the detection device is capable of detecting and distinguishing each detection product and each label in the same detection product.

66. The method of claim 65, wherein the first and second labels are detected and distinguished by passing the microspheres across or through the light.

67. The method of claim 65, wherein the first and second labels are detected and distinguished by passing the light across or through the microspheres.

68. The method of claim 65, wherein microspheres are on a two-dimensional surface.

69. The method of claim 58, wherein the binding members are selected from a group consisting of cofactors, receptors, receptor ligands, proteins, peptides, protein domains, oligonucleotides, transcription factors, nucleic acids, small molecules, and small compounds.

70. The method of claim 58, wherein the agent or agents to be screened are selected from a group consisting of cofactors, receptors, receptor ligands, proteins, peptides, protein domains, oligonucleotides, transcription factors, nucleic acids, small molecules, and small compounds.

71. The method of claim 58, wherein the binding members are cofactors and the agent or agents to be screened are receptors.

72. The method of claim 71, wherein the cofactors are coactivators, corepressors, or a combination of coactivators and corepressors.

73. The method of claim 71, wherein the receptor or receptors are orphan receptors.

74. The method of claim 71, wherein the receptor or receptors are nuclear receptors.

75. A method of screening, in single assay, for an agent that modulates binding between two or more first members of a binding pair and one or more second members of a binding pair, comprising
(a) contacting two or more first members and one or more second members of the binding pairs and the modulating agent, under conditions that allow formation of binding pairs, wherein the first members are coupled directly or indirectly to a set of microspheres labeled with a set of first labels; wherein each first label is specific for one first member; wherein the second member is coupled directly or indirectly to a second label; and wherein the second label is specific for one second member;
(b) detecting the presence of the first specific labels and the presence or absence of the second specific labels in a plurality of detection products, wherein each detection product comprises one microsphere;
(c) comparing, to a control, the relative amount of each second label associated with each first label in the detection products, wherein the control lacks the agent to be screened,
a greater or lesser amount of each second label associated with each first label in the detection products, indicating an agent that modulates binding between the specific first member, identified by the specific first label, and the specific second member, identified by the specific second label.

76. The method of claim 75, further comprising creating a modulating profile for each agent to be screened, wherein the modulating profile indicates the relative modulation of binding between each first member and each second member of the binding pairs, and comparing the modulating profile for the agent to be screened to a selected profile.

77. The method of claim 75, wherein the first and second labels are radiolabels, dyes, fluorescent labels, or a combination thereof.

78. The method of claim 75, wherein the microspheres contain the first labels.

79. The method of claim 75, wherein one or more detection products further comprises a second label.

80. The method of claim 75, wherein the microspheres are strepavidin-coated and wherein the first members are biotinylated.

81. The method of claim 75, wherein the microspheres are carboxylated and wherein the first members each have one or more free amine groups.

82. The method of claim 75, wherein the microspheres are maleimide-coated and wherein the first members each have one or more sulfhydryl reactive entities.

83. The method of claim 75, wherein the first and second labels are detected and distinguished with a detection device with a light source, wherein the detection device is capable of detecting and distinguishing each detection product and each label in the same detection product.

84. The method of claim 83, wherein the first and second labels are detected and distinguished by passing the microspheres across or through the light.

85. The method of claim 83, wherein the first and second labels are detected and distinguished by passing the light across or through the microspheres.

86. The method of claim 83, wherein microspheres are on a two-dimensional surface.

87. The method of claim 75, wherein the first members are selected from a group consisting of cofactors, receptors, receptor ligands, proteins, peptides, protein domains, oligonucleotides, transcription factors, nucleic acids, small molecules, and small compounds.

88. The method of claim 75, wherein the second members are selected from a group consisting of cofactors, receptors, receptor ligands, proteins, peptides, protein domains, oligonucleotides, transcription factors, nucleic acids, small molecules, and small compounds.

89. The method of claim 75, wherein the agent to be screened is selected from a group consisting of cofactors, receptors, receptor ligands, proteins, peptides, protein domains, oligonucleotides, transcription factors, nucleic acids, small molecules, and small compounds.

90. The method of claim 75, wherein the first members are cofactors, the second member or members are receptors.

91. The method of claim 90, wherein the cofactors are coactivators, corepressors, or a combination of coactivators and corepressors.

92. The method of claim 90, wherein the receptor or receptors are orphan receptors.

93. The method of claim 90, wherein the receptor or receptors are nuclear receptors.

94. A method of screening, in a single assay, for an agent that modulates binding between the members of two or more binding complexes, comprising
(a) contacting two or more first members, one or more second members, and a third member of the binding complexes with the agent to be screened, under conditions that allow formation of binding complexes, wherein each binding complex comprises one first and one second member of the binding pair; wherein the first members are coupled directly or indirectly to a set of microspheres labeled with a set of first labels; wherein each first label is specific for one first member; wherein the second member is coupled directly or indirectly to a second label; and wherein the second label is specific for one second member;
(b) detecting the presence of the first specific labels and the presence or absence of the second specific labels in a plurality of detection products, wherein each detection product comprises one microsphere; and
(c) comparing, to a control, the relative amount of each second label associated with each first label in the detection products, wherein the control lacks the agent to be screened,
a greater or lesser amount of each second label associated with each first label in the detection products, indicating an agent that modulates binding between the specific first member, identified by the specific first label, and the specific second member, identified by the specific second label, in the presence of the third member.

95. The method of claim 94, further comprising creating a modulating profile for each agent to be screened, wherein the modulating profile indicates the relative modulation of binding between each first member and each second member of the binding pairs in the presence of the third member, and comparing the modulating profile for the agent to be screened to a selected profile.

96. The method of claim 94, wherein the binding complex further comprises the third member of the binding complex.

97. The method of claim 94, wherein the third member of the binding complex potentiates or attenuates binding between the first member and second member of the binding complex.

98. The method of claim 94, wherein the first and second labels are radiolabels, dyes, fluorescent labels, or a combination thereof.

99. The method of claim 94, wherein the microspheres contain the first labels.

100. The method of claim 94, wherein one or more detection products further comprise a second label.

101. The method of claim 94, wherein the microspheres are strepavidin-coated and wherein the first members are biotinylated.

102. The method of claim 94, wherein the microspheres are carboxylated and wherein the first members each have one or more free amine groups.

103. The method of claim 94, wherein the microspheres are maleimide-coated and wherein the first members each have one or more sulfhydryl reactive entities.

104. The method of claim 94, wherein the first and second labels are detected and distinguished with a detection device with a light source, wherein the detection device is capable of detecting and distinguishing each detection product and each label in the same detection product.

105. The method of claim 104, wherein the first and second labels are detected and distinguished by passing the microspheres across or through the light.

106. The method of claim 104, wherein the first and second labels are detected and distinguished by passing the light across or through the microspheres.

107. The method of claim 104, wherein microspheres are on a two-dimensional surface.

108. The method of claim 94, wherein the first members are selected from a group consisting of cofactors, receptors, receptor ligands, proteins, peptides, protein domains, oligonucleotides, transcription factors, nucleic acids, small molecules, and small compounds.

109. The method of claim 94, wherein the second members are selected from a group consisting of cofactors, receptors, receptor ligands, proteins, peptides, protein domains, oligonucleotides, transcription factors, nucleic acids, small molecules, and small compounds.

110. The method of claim 94, wherein the agent to be screened is selected from a group consisting of cofactors, receptors, receptor ligands, proteins, peptides, protein domains, oligonucleotides, transcription factors, nucleic acids, small molecules, and small compounds.

111. The method of claim 94, wherein the first members are cofactors, the second member or members are receptors and the third member is a receptor ligand.

112. The method of claim 111, wherein the cofactors are coactivators, corepressors, or a combination of coactivators and corepressors.

113. The method of claim 111, wherein the receptor or receptors are orphan receptors.

114. The method of claim 111, wherein the receptor or receptors are nuclear receptors.

115. A set of microspheres coupled with a set of cofactors, wherein the microspheres are labeled with a label specific for each cofactor of the set.

116. The set of microspheres of claim 115, wherein the cofactors comprise coactivators, corepressors, or both coactivators and corepressors.

117. The set of microspheres of claim 115, wherein the cofactors are nuclear receptor cofactors.

118. The set of microspheres of claim 115, wherein the coactivators comprise a five amino acid motif having, from amino to carboxy end, a leucine residue, two additional amino acid residues of any identity, and two leucine residues.

119. The set of microspheres of claim 115, wherein the corepressors comprise a five amino acid motif having, from amino to carboxy end, a leucine residue, two additional amino acid residues, and two isoleucine residues.


SEQUENCE LISTING (44 sequences)

SEQ ID NO: 1 - protein, 17 aa, Artificial Sequence
  Description of Artificial Sequence/note = synthetic construct
YSQTSHKLVQLLTTTAE

SEQ ID NO: 2 - protein, 25 aa, Artificial Sequence
  Description of Artificial Sequence/note = synthetic construct
CPSSHSSLTERHKILHRLLQEGSPS

SEQ ID NO: 3 - protein, 25 aa, Artificial Sequence
  Description of Artificial Sequence/note = synthetic construct
LDASKKKESKDHQLLRYLLDKDEKD

SEQ ID NO: 4 - protein, 23 aa, Artificial Sequence
  Description of Artificial Sequence/note = synthetic construct
NLVPDAASKHKQLSELLRGGSGS

SEQ ID NO: 5 - protein, 23 aa, Artificial Sequence
  Description of Artificial Sequence/note = synthetic construct
ADPEKRKLIQQQLVLLLHAHKCQ

SEQ ID NO: 6 - protein, 23 aa, Artificial Sequence
  Description of Artificial Sequence/note = synthetic construct
SVQPPRSISPSALQDLLRTLKSP

SEQ ID NO: 7 - protein, 27 aa, Artificial Sequence
  Description of Artificial Sequence/note = synthetic construct
PVSSMAGNTKNHPMLMNLLKDNPAQDF

SEQ ID NO: 8 - protein, 27 aa, Artificial Sequence
  Description of Artificial Sequence/note = synthetic construct
GSTHGTSLKEKHKILHRLLQDSSSPVD

SEQ ID NO: 9 - protein, 27 aa, Artificial Sequence
  Description of Artificial Sequence/note = synthetic construct
SNMHGSLLQEKHRILHKLLQNGNSPAE

SEQ ID NO: 10 - protein, 27 aa, Artificial Sequence
  Description of Artificial Sequence/note = synthetic construct
PVSPQDFSFSKNGLLSRLLRQNQDSYL

SEQ ID NO: 11 - protein, 27 aa, Artificial Sequence
  Description of Artificial Sequence/note = synthetic construct
RTPLGSAMLRPNPILARLLRAHGAPEP

SEQ ID NO: 12 - protein, 27 aa, Artificial Sequence
  Description of Artificial Sequence/note = synthetic construct
SLGMVQDAASKHKQLSELLRSGSSPNL

SEQ ID NO: 13 - protein, 25 aa, Artificial Sequence
  Description of Artificial Sequence/note = synthetic construct
ENQRGPLESKGHKKLLQLLTCSSDD

SEQ ID NO: 14 - protein, 25 aa, Artificial Sequence
  Description of Artificial Sequence/note = synthetic construct
DGQSRLHDSKGQTKLLQLLTTKSDQ

SEQ ID NO: 15 - protein, 25 aa, Artificial Sequence
  Description of Artificial Sequence/note = synthetic construct
EHRSWARESKSFNVLKQLLLSENCV

SEQ ID NO: 16 - protein, 25 aa, Artificial Sequence
  Description of Artificial Sequence/note = synthetic construct
EKRRREQEWKYLEELAELLSANISD

SEQ ID NO: 17 - protein, 25 aa, Artificial Sequence
  Description of Artificial Sequence/note = synthetic construct
RISQSTFNNPRPGQLGRLLPNQNLP

SEQ ID NO: 18 - protein, 25 aa, Artificial Sequence
  Description of Artificial Sequence/note = synthetic construct
KKKLVSQNNEQGSTLRDLLTTTAGK

SEQ ID NO: 19 - protein, 25 aa, Artificial Sequence
  Description of Artificial Sequence/note = synthetic construct
HKEEDADTKQVYFYLFKLLRKSILQ

SEQ ID NO: 20 - protein, 25 aa, Artificial Sequence
  Description of Artificial Sequence/note = synthetic construct
STALLYSKRLITYRLRHLLRARCDA

SEQ ID NO: 21 - protein, 25 aa, Artificial Sequence
  Description of Artificial Sequence/note = synthetic construct
GISPLKPGTVSQQALQNLLRTLRSP

SEQ ID NO: 22 - protein, 25 aa, Artificial Sequence
  Description of Artificial Sequence/note = synthetic construct
GPHGSQNRPLLRNSLDDLLGPPSNA

SEQ ID NO: 23 - protein, 25 aa, Artificial Sequence
  Description of Artificial Sequence/note = synthetic construct
KKKSTGQGVIDKDSLGPLLLQALDG

SEQ ID NO: 24 - protein, 25 aa, Artificial Sequence
  Description of Artificial Sequence/note = synthetic construct
KKKDVHQDSIVLTYLEGLLMHQAAG

SEQ ID NO: 25 - protein, 25 aa, Artificial Sequence
  Description of Artificial Sequence/note = synthetic construct
KKKRPQNANYPRSILTSLLLNSSQS

SEQ ID NO: 26 - protein, 25 aa, Artificial Sequence
  Description of Artificial Sequence/note = synthetic construct
TKFVNLYTRERQDRLAVLLPGRHPC

SEQ ID NO: 27 - protein, 25 aa, Artificial Sequence
  Description of Artificial Sequence/note = synthetic construct
KKKGQSDERALLDQLHTLLSNTDAT

SEQ ID NO: 28 - protein, 25 aa, Artificial Sequence
  Description of Artificial Sequence/note = synthetic construct
QEQLSPKKKENNALLRYLLDRDDPS

SEQ ID NO: 29 - protein, 25 aa, Artificial Sequence
  Description of Artificial Sequence/note = synthetic construct
QEPVSPKKKENALLRYLLDKDDTKD

SEQ ID NO: 30 - protein, 25 aa, Artificial Sequence
  Description of Artificial Sequence/note = synthetic construct
SLQNLKNLGESATLRSLLLNPHLRQ

SEQ ID NO: 31 - protein, 25 aa, Artificial Sequence
  Description of Artificial Sequence/note = synthetic construct
SKNSKLNSHQKVTLLQLLLGHKNEE

SEQ ID NO: 32 - protein, 25 aa, Artificial Sequence
  Description of Artificial Sequence/note = synthetic construct
SGSEIENLLERRTVLQLLLGNPTKG

SEQ ID NO: 33 - protein, 25 aa, Artificial Sequence
  Description of Artificial Sequence/note = synthetic construct
LERNNIKQAANNSLLLHLLKSQTIP

SEQ ID NO: 34 - protein, 25 aa, Artificial Sequence
  Description of Artificial Sequence/note = synthetic construct
MVDSVRKGKQDSTLLASLLQSFSSR

SEQ ID NO: 35 - protein, 25 aa, Artificial Sequence
  Description of Artificial Sequence/note = synthetic construct
EKDLRCYGVASSHLKTLLKKSKVKD

SEQ ID NO: 36 - protein, 25 aa, Artificial Sequence
  Description of Artificial Sequence/note = synthetic construct
ALHLAVIHQHEPFLDFLLGFSAGTE

SEQ ID NO: 37 - protein, 25 aa, Artificial Sequence
  Description of Artificial Sequence/note = synthetic construct
EQNVSSLNEREQELHNLLEVVSQCC

SEQ ID NO: 38 - protein, 26 aa, Artificial Sequence
  Description of Artificial Sequence/note = synthetic construct
GQVPRTHRLITLADHICQIITQDFAR

SEQ ID NO: 39 - protein, 25 aa, Artificial Sequence
  Description of Artificial Sequence/note = synthetic construct
GHSFADPASNLGLEDIIRKALMGSF

SEQ ID NO: 40 - protein, 26 aa, Artificial Sequence
  Description of Artificial Sequence/note = synthetic construct
PGVKGHQRVVTLAQHISEVITQDYTR

SEQ ID NO: 41 - protein, 26 aa, Artificial Sequence
  Description of Artificial Sequence/note = synthetic construct
SQAVQEHASTNMGLEAIIRKALMGKY

SEQ ID NO: 42 - protein, 25 aa, Artificial Sequence
  Description of Artificial Sequence/note = synthetic construct
PAGPRGMGTALKLLLGAGAVAYGVR

SEQ ID NO: 43 - protein, 25 aa, Artificial Sequence
  Description of Artificial Sequence/note = synthetic construct
AIFFNRIGGVQQDTILAEGLHFRIP

SEQ ID NO: 44 - protein, 9 aa, Artificial Sequence
  Description of Artificial Sequence/note = synthetic construct
MKKGHHHHG